(19) United States
(12) Patent Application Publication
(10) Pub. No.: US 2019/0069778 A1
(43) Pub. Date: Mar. 7, 2019

(54) PORTABLE TELE-MONITORING HEALTH CARE SYSTEM AND METHOD

(71) Applicants: Seyed Mohammad Pourhoseini, Tehran (IR); Khalil Torkan, Tehran (IR)

(72) Inventors: Seyed Mohammad Pourhoseini, Tehran (IR); Khalil Torkan, Tehran (IR)

(21) Appl. No.: 15/696,826

(22) Filed: Sep. 6, 2017

(51) Int. Cl. A61B 5/00; G16H 10/60

(52) U.S. Cl. CPC A61B 5/0022; G16H 40/60; G16H 10/60; A61B 5/0006

(57) ABSTRACT

The embodiments herein provide a portable health care tele-monitoring device for intensive care unit of hospital, inside ambulance/vehicle or home. The device facilitates real time monitoring of patient's health. Patient information gathered using the device comprises personal details, health records and real time medical parameters of the patient. A blood parameter measurement device, external devices and vital sign devices are connected to the device using various ports and connectors provided in the device. The device facilitates transmission of patient information to a central location such as hospital, personal clinic of a doctor or health care centre and receiving medical prescription remotely over communication network from doctor/medical expert based on patient information captured by the device. Optionally physical copy of vital signs and blood parameters is printed using the device. A user interface is provided to enable patient/user for communicating with doctor/medical expert whenever needed.

### BACKGROUND

### Technical Field

The embodiments herein are generally related to a field of health care. The embodiments herein are particularly related to a portable, remote tele-monitoring health care device. The embodiments herein are more particularly related to a system, device and method for monitoring vital signs and capturing data from a patient remotely using tele-monitoring techniques.

### Description of the Related Art

The monitoring of a patient in places such as an intensive care unit of a hospital, ambulance or home requires a more vigilant surveillance by one or more nurses. Scrutiny of the vital signs and blood factors is significant for the doctors to make better decisions about an accurate medical condition of the patient. Further, physiological monitoring is also used in surgical operation, patients with an unstable situation and those who are confined to bed in hospital. Several monitoring systems are known in the art for aiding nurses and doctors.

Health monitoring involves the use of telecommunications and information technology for the purpose of monitoring a patient's health. The use of tele-monitoring health care devices is grown widely all over the world due to several factors such as reduction in costs of health care, an increase in the number of aging and chronically ill population and the increased coverage of health care to remote, rural and small regions. Tele-monitoring devices help to solve many problems such as shortage of qualified health care providers, long distances to reach hospitals, higher travel time and crowded hospitals.

However, all currently available health monitoring devices are rigid in nature and are capable of providing only first aid treatments to the patients. Also, these devices are quite bulky and hence not portable enough. Further, the devices do not offer monitoring of multiple health parameters and hence provide insufficient data to the doctors for proper diagnosis. An exact location detection and accurate arrival time of the ambulance/vehicle is very much difficult in the conventional tele-monitoring devices that are deployed in ambulances/vehicles. This results in delay in providing medical care to the patients in timely manner. Additionally, the conventional devices are not capable of instantly transmitting video/audio to the doctors to suggest medical first-aid instructions or give online advice (for example, heart electroshock). Thus, the doctor needs to examine the patient physically and which is either cumbersome to the patient or is life threatening in emergency situations.

Hence, there is a need for developing a light weight and portable tele-monitoring health care device that is used for monitoring a patient in critical areas such as an intensive care unit of a hospital, ambulance or home. There is also a need for a device that facilitates a checking of a plurality of vital signs and capturing data from a patient remotely using tele-monitoring techniques. Further, there is a need for a device that helps the patients in early diagnosis and treatment of diseases by offering remote and easy access to expert medical specialists. Still further, there is a need for a device that helps the doctors to prescribe medical prescriptions remotely after checking vital signs of the patient over internet.

The above mentioned shortcomings, disadvantages and problems are addressed herein and which will be understood by reading and studying the following specification.

### OBJECTS OF THE EMBODIMENTS

The primary object of the embodiments herein is to provide a light weight and portable tele-monitoring health care device for monitoring a patient in critical areas such as an intensive care unit of a hospital, ambulance or home, etc.

Another object of the embodiments herein is to provide a portable tele-monitoring health care device that facilitates a checking of a plurality of vital signs and capturing data from a patient remotely using tele-monitoring techniques.

Yet another object of the embodiments herein is to provide a portable tele-monitoring health care device that helps the patients to have early diagnosis and treatment of diseases by offering easy access to expert medical specialists.

Yet another object of the embodiments herein is to provide a portable tele-monitoring health care device to provide a patient with video telephony/conferencing and online consultation with the doctors.

Yet another object of the embodiments herein is to provide a portable tele-monitoring health care device that helps in reducing the doctor/physician fees and cost of medicines.

Yet another object of the embodiments herein is to provide a portable tele-monitoring health care device to with user friendly graphical user interfaces to enable the patients/operators to have easier health check and self-health management.

Yet another object of the embodiments herein is to provide a portable tele-monitoring health care device that facilitates a checking of plurality of vital signs such as ECG, SpO₂, Respiration, NIBP, IBP, Temperature, CO₂ and blood factor such as PCG, Glucose, Cholesterol, Triglycerides, HDL etc.

Yet another object of the embodiments herein is to provide a portable tele-monitoring health care device for ambulances to enable a real time tracking of an ambulance and also in determining an arrival time of the ambulance accurately.

Yet another object of the embodiments herein is to provide a portable tele-monitoring health care device configured for transmitting data related to the vital signs of the patient using 2G, 3G or Wi-Fi networks.

Yet another object of the embodiments herein is to provide a portable tele-monitoring health care device configured for maintaining the electronic health records (EHR) of the patient.

Yet another object of the embodiments herein is to provide a portable tele-monitoring health care device that offers expert medical consultation through video telephony and as well as chat based interface thereby drastically reducing the consultation charges to be paid by the patient.

Yet another object of the embodiments herein is to provide a portable tele-monitoring health care device for the hospitals or health care organizations for easy management of large number of patients.

These and other objects and advantages of the embodiments herein will become readily apparent from the following detailed description taken in conjunction with the accompanying drawings.

### SUMMARY

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The various embodiments herein provide a portable tele-monitoring health care device. The device comprises a display unit configured for displaying a list of plurality of patients registered with the device, patient information and device information. The patient information comprises one or more personal details of the patient, one or more health records of the patient and one or more real time medical parameters of the patient. The device information comprises a plurality of device configurable parameters and an instruction manual for operating the device. The display unit comprises a LCD/LED screen enclosed within a housing. The housing comprises a base portion, a top portion, a first lateral side and a second lateral side. The device also comprises a first panel provided in the base portion of the housing configured for accommodating a thermal printer, a printer output port, a blood parameter measurement device and a blood parameter measurement device port. The thermal printer is configured for printing a plurality of vital signs of the patient. The printer output port is configured for connecting external printer. The blood parameter measurement device is configured for measuring a plurality of blood parameters of the patient. The plurality of blood parameters of the patient includes cholesterol, high-density lipoprotein (HDL), low-density lipoprotein (LDL), triglycerides and blood glucose. The device further comprises a second panel provided in the first lateral side of the housing configured for accommodating a headphone connector, a Video Graphics Array (VGA) connector, one or more USB ports, one or more USB to serial ports and a USB hub. The one or more USB ports, USB to serial ports and the USB hub are configured for connecting one or more external devices to the tele-monitoring device. The one or more external devices comprise a stethoscope, a telecommunication device and a networking device. The device still further comprises a third panel provided in the second lateral side of the housing configured for accommodating one or more connectors for connecting one or more vital sign devices to the tele-monitoring device. One or more vital sign devices are configured for measuring the plurality of vital signs of the patient. The plurality of vital signs of the patient comprise electro cardio gram (ECG), blood oxygen saturation level (SPO₂) level, respiration rate, non-invasive blood pressure (NIBP), invasive blood pressure (IBP), temperature, carbon dioxide (CO₂) and phonocardiogram (PCG). The device still further comprises one or more cameras provided in the top portion of the housing configured for capturing one or more images of the patient or for facilitating real time video conferencing between the patient and a doctor. The device still further comprises a central processing unit (CPU) provided within the housing configured for controlling operation of the portable tele-monitoring device health care and for storing the list of plurality of patients, patient information and device information in a database.

According to an embodiment herein, the device is further configured for enabling a user/attendant/medical service provider to access the device by logging into the device using an exclusive username and password. The one or more users/attendants/service providers are registered with the device using the username and password.

According to an embodiment herein, the device further comprises one or more tabs provided in the first panel. The one or more tabs are configured for performing one or more operations of the device when the one or more tabs are pressed by an attendant. The one or more operations performed comprise powering ON/OFF the device, displaying a main menu page of the device, displaying a previously accessed page by the attendant, downloading and displaying one or more real time medical parameters of the patient and controlling the device volume.

According to an embodiment herein, the device further comprises a connectivity module configured for establishing a connection between the portable tele-monitoring health care device and a central location or a central server. The central location comprises a hospital, a personal clinic of a doctor/medical expert or a health care centre. The central server is provided at any one of a location selected from a group consisting of a hospital, a personal clinic of a doctor/medical expert or a health care centre. The communication between the portable health care tele-monitoring device and a central location/server is established using Wi-Fi, Bluetooth or GPRS based connectivity.

According to an embodiment herein, the device further comprises a control board configured for enabling a communication connection of the portable tele-monitoring health care device with an external keypad, LED indicator/alarm, a power source and a global positioning system.

According to an embodiment herein, the device further comprises a connector port configured for enabling a connection of the portable tele-monitoring health care device with a high-definition multimedia interface (HDMI) and a microphone (mic).

According to an embodiment herein, the device is further configured for use in an intensive care unit of a hospital, inside an ambulance/vehicle or within a home.

According to an embodiment herein, the device further comprises a location detection module configured for detecting a real time location of the device. The real time location of the device is transmitted to the central location.

According to an embodiment herein, the device is further configured for enabling the doctors/medical experts to write/share a prescription remotely based on the one or more health records of the patient and the one or more real time medical parameters of the patient monitored using the health care monitoring device.

According to an embodiment herein, the device further comprises a touch screen keypad displayed using the display unit configured for enabling the user/attendant/medical service provider to login into the device using the exclusive username and password.

According to an embodiment herein, the device is further configured for providing a user interface to the patient/user for communicating with the doctor/medical expert whenever needed.

According to an embodiment herein, a method of using a portable tele-monitoring health care device is provided. The method comprises the steps of deploying a portable health care tele-monitoring device in an intensive care unit of a hospital, or inside an ambulance/vehicle or within a home for monitoring a patient's health. A user or a patient is logged-in into the portable health care tele-monitoring device by entering exclusive username and password to access patient information and device information. The patient information comprises one or more personal details of the patient, one or more health records of the patient and one or more real time medical parameters of the patient. The device information comprises a plurality of device configurable parameters and an instruction manual for operating the device. A blood parameter measurement device, one or more external devices and one or more vital sign devices are connected to the portable tele-monitoring health care device through a blood parameter measurement device port, one or more USB to serial ports, USB hub and one or more connectors provided in the device. A plurality of vital signs and one or more blood parameters of the patient are checked/monitored with the one or more vital sign devices and the blood parameter measuring device. The results of the plurality of vital signs and one or more blood parameters are displayed on the display unit of the portable tele-monitoring health care device. The patient information is transmitted to a central server in a central location such as a hospital, a personal clinic of a doctor/medical expert or a health care centre. Video conferencing/chat is enabled with the doctor/medical expert using one or more cameras or imaging devices provided in the portable tele-monitoring health care device. A medical prescription is received remotely over a communication network from the doctor/medical expert based on patient information captured by the device.

According to an embodiment herein, the method further comprises tracking the location of the ambulance with the portable tele-monitoring health care device in real time using a location determination module provided in the portable tele-monitoring health care device.

According to an embodiment herein, the method further comprises providing a user interface to the patient/user for communicating with the doctor/medical expert whenever needed.

According to an embodiment herein, the method further comprises optionally printing a physical copy of the plurality of vital signs and one or more blood parameters using a thermal printer or external printer attached with the portable health care tele-monitoring device.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### BRIEF DESCRIPTION OF THE DRAWINGS

The other objects, features and advantages will occur to those skilled in the art from the following description of the preferred embodiment and the accompanying drawings in which:

FIG. 1 illustrates a front side view of a portable tele-monitoring health care device, according to one embodiment herein.

FIG. 2 illustrates a back side view of the portable tele-monitoring health care device, according to one embodiment herein.

FIG. 3 illustrates a block circuit diagram indicating the various internal components of the portable tele-monitoring health care device, according to one embodiment herein.

FIG. 4 illustrates a screen shot of a login screen provided by the tele-monitoring health care device, according to one embodiment herein.

FIG. 5 illustrates a screen shot of a main/home screen provided by the tele-monitoring health care device, according to one embodiment herein.

FIG. 6 illustrates a screen shot of a patient information display screen provided by the tele-monitoring health care device, according to one embodiment herein.

FIG. 7 illustrates a screen shot of a patient's vital signs measurement display screen provided by the tele-monitoring health care device, according to one embodiment herein.

FIG. 8 illustrates a screen shot of an ECG signal measurement and record display screen provided by the tele-monitoring health care device, according to one embodiment herein.

FIG. 9 illustrates a screen shot of a respiration measurement display screen provided by the tele-monitoring health care device, according to one embodiment herein.

FIG. 10 illustrates a screen shot of a blood oxygen saturation measurement display screen provided by the tele-monitoring health care device, according to one embodiment herein.

FIG. 11 illustrates a screen shot of an invasive blood pressure measurement display screen provided by the tele-monitoring health care device, according to one embodiment herein.

FIG. 12 illustrates a screen shot of a noninvasive blood pressure measurement display screen provided by the tele-monitoring health care device, according to one embodiment herein.

FIG. 13 illustrates a screen shot of a patient's body temperature measurement display screen provided by the tele-monitoring health care device, according to one embodiment herein.

FIG. 14 illustrates a screen shot of a CO₂ exhalation measurement display screen provided by the tele-monitoring health care device, according to one embodiment herein.

FIG. 15 illustrates a screen shot of a blood glucose measurement display screen provided by the tele-monitoring health care device, according to one embodiment herein.

FIG. 16 illustrates a screen shot indicating a four types of fats display screen provided by the tele-monitoring health care device, according to one embodiment herein.

FIG. 17 illustrates a screen shot of a PCG recording display screen provided by the tele-monitoring health care device, according to one embodiment herein.

FIG. 18 illustrates a screen shot of a medical image taken by the tele-monitoring health care device, according to one embodiment herein.

FIG. 19 illustrates a screen shot of a prescription page provided by the tele-monitoring health care device, according to one embodiment herein.

FIG. 20 illustrates a screen shot of a page indicating all unsaved vital signs of the patient, according to one embodiment herein.

FIG. 21 illustrates a screen shot of a page displaying data captured and stored from a patient as per date and time of visit, according to one embodiment herein.

FIG. 22 illustrates a screen shot of a page indicating various print or printer settings available in the tele-monitoring health care device, according to one embodiment herein.

FIG. 23 illustrates a screen shot of a page indicating date and time settings, according to one embodiment herein.

FIG. 24 illustrates a screen shot of a page indicating Wi-Fi settings, according to one embodiment herein.

FIG. 25 illustrates a screen shot of a page indicating power on/off and Bluetooth settings, according to one embodiment herein.

FIG. 26 illustrates a screen shot of a page indicating overall settings, according to one embodiment herein.

FIG. 27 illustrates a screen shot of a page indicating Ethernet settings, according to one embodiment herein.

FIG. 28 illustrates a screen shot of a page indicating Bluetooth settings, according to one embodiment herein.

FIG. 29 illustrates a screen shot of a page indicating mobile data settings, according to one embodiment herein.

FIG. 30 illustrates a screen shot of a page indicating GPS settings, according to one embodiment herein.

FIG. 31 illustrates a screen shot of a page indicating general settings, according to one embodiment herein.

FIG. 32 illustrates a screen shot of a page displaying a help screen, according to one embodiment herein.

FIG. 33 illustrates a screen shot of a device information display screen, according to one embodiment herein.

FIG. 34 illustrates a front view of a blood parameter measuring device provided with the portable health care tele-monitoring device, according to one embodiment herein.

FIG. 35 illustrates various blood parameter measuring stripes used with the blood parameter measuring device, according to one embodiment herein.

FIG. 36 illustrates a pictorial representation of the process steps followed in code testing for the blood parameter measuring device, according to one embodiment herein.

FIG. 37 illustrates a pictorial representation of the process steps followed in measuring plurality of blood parameters using the blood parameter measurement device, according to one embodiment herein.

FIG. 38 illustrates a block diagram of a portable health care tele-monitoring system with the devices used in ambulances, according to one embodiment herein.

FIG. 39 illustrates a flow chart explaining a method of using the portable health care tele-monitoring device, according to one embodiment herein.

Although the specific features of the present invention are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a portable tele-monitoring health care device. The device comprises a display unit configured for displaying a list of plurality of patients registered with the device, patient information and device information. The patient information comprises one or more personal details of the patient, one or more health records of the patient and one or more real time medical parameters of the patient. The device information comprises a plurality of device configurable parameters and an instruction manual for operating the device. The display unit comprises a LCD/LED screen enclosed within a housing. The housing comprises a base portion, a top portion, a first lateral side and a second lateral side. The device also comprises a first panel provided in the base portion of the housing configured for accommodating a thermal printer, a printer output port, a blood parameter measurement device and a blood parameter measurement device port. The thermal printer is configured for printing a plurality of vital signs of the patient. The printer output port is configured for connecting external printer. The blood parameter measurement device is configured for measuring a plurality of blood parameters of the patient. The plurality of blood parameters of the patient includes cholesterol, high-density lipoprotein (HDL), low-density lipoprotein (LDL), triglycerides and blood glucose. The device further comprises a second panel provided in the first lateral side of the housing configured for accommodating a headphone connector, a Video Graphics Array (VGA) connector, one or more USB ports, one or more USB to serial ports and a USB hub. The one or more USB ports, USB to serial ports and the USB hub are configured for connecting one or more external devices to the tele-monitoring device. The one or more external devices comprise a stethoscope, a telecommunication device and a networking device. The device still further comprises a third panel provided in the second lateral side of the housing configured for accommodating one or more connectors for connecting one or more vital sign devices to the tele-monitoring device. One or more vital sign devices are configured for measuring the plurality of vital signs of the patient. The plurality of vital signs of the patient comprise electro cardio gram (ECG), blood oxygen saturation level (SPO₂) level, respiration rate, non-invasive blood pressure (NIBP), invasive blood pressure (IBP), temperature, carbon dioxide (CO₂) and phonocardiogram (PCG). The device still further comprises one or more cameras provided in the top portion of the housing configured for capturing one or more images of the patient or for facilitating real time video conferencing between the patient and a doctor. The device still further comprises a central processing unit (CPU) provided within the housing configured for controlling operation of the portable tele-monitoring device health care and for storing the list of plurality of patients, patient information and device information in a database.

According to an embodiment herein, the device is further configured for enabling a user/attendant/medical service provider to access the device by logging into the device using an exclusive username and password. The one or more users/attendants/service providers are registered with the device using the username and password.

According to an embodiment herein, the device further comprises one or more tabs provided in the first panel. The one or more tabs are configured for performing one or more operations of the device when the one or more tabs are pressed by an attendant. The one or more operations performed comprise powering ON/OFF the device, displaying a main menu page of the device, displaying a previously accessed page by the attendant, downloading and displaying one or more real time medical parameters of the patient and controlling the device volume.

According to an embodiment herein, the device further comprises a connectivity module configured for establishing a connection between the portable tele-monitoring health care device and a central location or a central server. The central location comprises a hospital, a personal clinic of a doctor/medical expert or a health care center. The central server is provided at any one of a location selected from a group consisting of a hospital, a personal clinic of a doctor/medical expert or a health care center. The communication between the portable health care tele-monitoring device and a central location/server is established using Wi-Fi, Bluetooth or GPRS based connectivity.

According to an embodiment herein, the device further comprises a control board configured for enabling a communication connection of the portable tele-monitoring health care device with an external keypad, LED indicator/alarm, a power source and a global positioning system.

According to an embodiment herein, the device further comprises a connector port configured for enabling a connection of the portable tele-monitoring health care device with a high-definition multimedia interface (HDMI) and a microphone (mic).

According to an embodiment herein, the device is further configured for use in an intensive care unit of a hospital, inside an ambulance/vehicle or within a home.

According to an embodiment herein, the device further comprises a location detection module configured for detecting a real time location of the device. The real time location of the device is transmitted to the central location.

According to an embodiment herein, the device is further configured for enabling the doctors/medical experts to write/share a prescription remotely based on the one or more health records of the patient and the one or more real time medical parameters of the patient monitored using the health care monitoring device.

According to an embodiment herein, the device further comprises a touch screen keypad displayed using the display unit configured for enabling the user/attendant/medical service provider to login into the device using the exclusive username and password.

According to an embodiment herein, the device is further configured for providing a user interface to the patient/user for communicating with the doctor/medical expert whenever needed.

According to an embodiment herein, a method of using a portable tele-monitoring health care device is provided. The method comprises the steps of deploying a portable health care tele-monitoring device in an intensive care unit of a hospital, or inside an ambulance/vehicle or within a home for monitoring a patient's health. A user or a patient is logged-in into the portable health care tele-monitoring device by entering exclusive username and password to access patient information and device information. The patient information comprises one or more personal details of the patient, one or more health records of the patient and one or more real time medical parameters of the patient. The device information comprises a plurality of device configurable parameters and an instruction manual for operating the device. A blood parameter measurement device, one or more external devices and one or more vital sign devices are connected to the portable tele-monitoring health care device through a blood parameter measurement device port, one or more USB to serial ports, USB hub and one or more connectors provided in the device. A plurality of vital signs and one or more blood parameters of the patient are checked/monitored with the one or more vital sign devices and the blood parameter measuring device. The results of the plurality of vital signs and one or more blood parameters are displayed on the display unit of the portable tele-monitoring health care device. The patient information is transmitted to a central server in a central location such as a hospital, a personal clinic of a doctor/medical expert or a health care centre. Video conferencing/chat is enabled with the doctor/medical expert using one or more cameras or imaging devices provided in the portable tele-monitoring health care device. A medical prescription is received remotely over a communication network from the doctor/medical expert based on patient information captured by the device.

According to an embodiment herein, the method further comprises tracking the location of the ambulance with the portable tele-monitoring health care device in real time using a location determination module provided in the portable tele-monitoring health care device.

According to an embodiment herein, the method further comprises providing a user interface to the patient/user for communicating with the doctor/medical expert whenever needed.

According to an embodiment herein, the method further comprises optionally printing a physical copy of the plurality of vital signs and one or more blood parameters using a thermal printer or external printer attached with the portable health care tele-monitoring device.

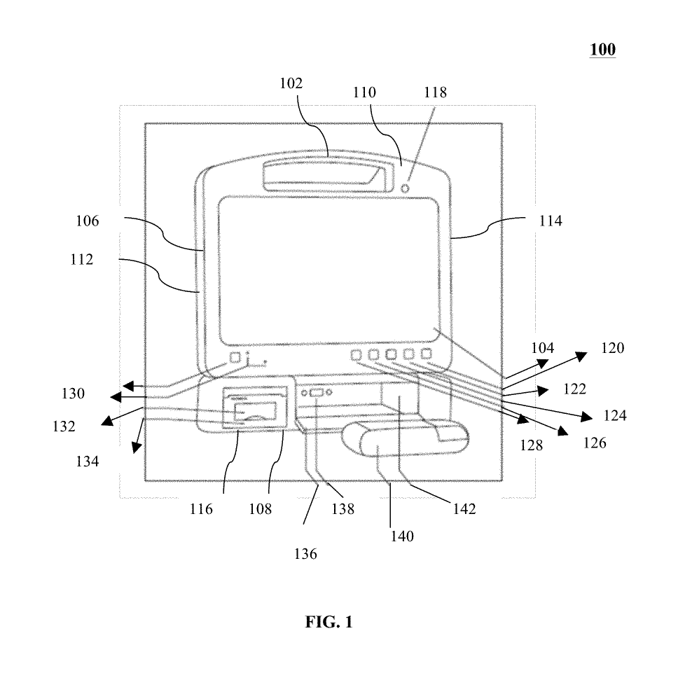

FIG. 1 illustrates a front view of a portable tele-monitoring health care device, according to one embodiment herein. With respect to FIG. 1, the device comprises a display unit 102 configured for displaying a list of plurality of patients registered with the device, patient information and device information. The patient information comprises one or more personal details of the patient, one or more health records of the patient and one or more real time medical parameters of the patient. The device information comprises a plurality of device configurable parameters and a instruction manual for operating the device. The display unit comprises the LCD/LED screen 104 deployed within the housing 106. The housing comprises the base portion 108, the top portion 110, the first lateral side 112 and the second lateral side 114. The first panel 116 is provided in the base portion 108 of the housing 106. The first panel 116 is configured for accommodating a thermal printer, a printer output port, a blood parameter measurement device and a blood parameter measurement device port. The thermal printer is configured for printing a plurality of vital signs of the patient. The printer output port is configured for connecting external printer. The blood parameter measurement device is configured for measuring a plurality of blood parameters of the patient. The plurality of blood parameters of the patient comprise cholesterol, HDL (high-density lipoprotein), LDL (low-density lipoprotein), triglycerides and blood glucose.

According to an embodiment herein, the second panel (not shown) is provided in the first lateral side 112 of the housing 106. The second panel is configured for accommodating a headphone connector, a Video Graphics Array (VGA) connector, a one or more USB to serial ports and a USB hub. The one or more USB to serial ports and the USB hub are configured for connecting one or more external devices to the tele-monitoring device. The one of more external devices comprise a stethoscope, a telecommunication device and a networking device.

According to an embodiment herein, the third panel (not shown) is provided in the second lateral side 114 of the housing 106. The third panel is configured for accommodating one or more connectors for capturing plurality of vital signs of the patient. The plurality of vital signs of the patient comprise electro cardio gram (ECG), blood oxygen saturation level (SPO₂) level, respiration rate, non-invasive blood pressure (NIBP), invasive blood pressure (IBP), temperature, carbon dioxide (CO₂) and phonocardiogram (PCG).

According to an embodiment herein, one or more cameras 118 are provided in the top portion 110 of the housing 106. The one or more cameras 118 are configured for capturing one or more images or for facilitating real time video conferencing between the patient and a doctor.

According to an embodiment herein, a central processing unit (CPU) (not shown) is provided in the housing. The CPU is configured for controlling operation of the portable health care tele-monitoring device and for storing the list of plurality of patients, patient information and device information in a database (not shown).

According to an embodiment herein, the device further comprises one or more tabs provided in the first panel 116. The one or more tabs are configured for performing one or more operations of the device when pressed by an attendant. The one or more operations performed comprise powering ON/OFF the device (120), displaying a main menu page of the device (122), displaying a previously accessed page by the attendant (124), downloading and displaying one or more real time medical parameters of the patient (126) and controlling the device volume (128). The front panel 116 further comprises two LED indicators (130) that show the situation of a battery used for powering the device and to indicate whether the device is on or off. The thermal printer (132) used for printing the patient's vital signs is also placed in the front panel 116. The front panel further comprises the printer output port (134) a base tray for placing the blood parameter measurement device (136), a USB cable connection from the LCD/LED monitor to the blood parameters measurement device (138), the blood parameter measuring device (140) and a box for inserting the blood parameter measuring device (142).

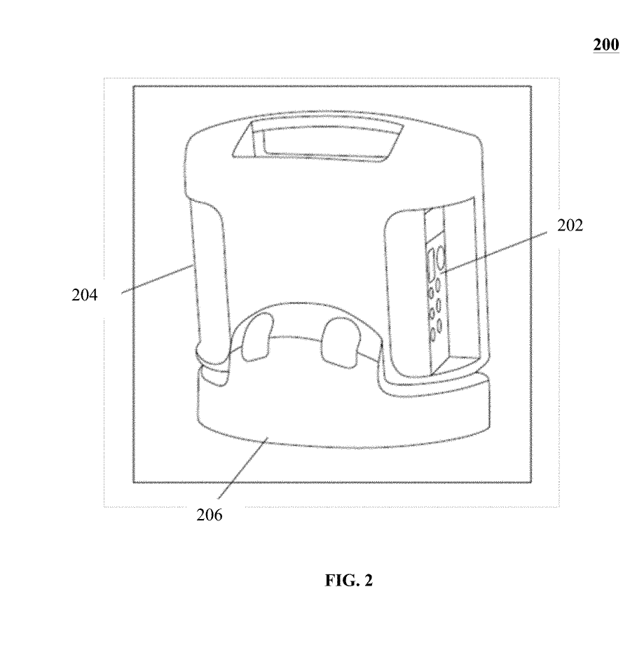

FIG. 2 illustrates a back view of the portable tele-monitoring health care device, according to one embodiment herein. With respect to FIG. 2, the second panel 202 and the third panel 204 is shown. The device further comprises a pedestal 206 configured for providing a support for placing the device.

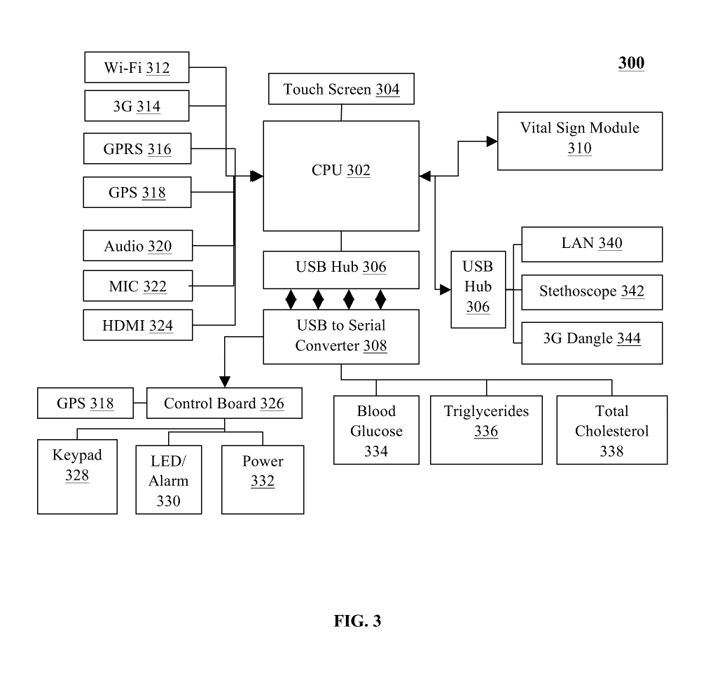

FIG. 3 illustrates a block diagram depicting various internal components of the tele-monitoring health care device, according to one embodiment herein. The device comprises the CPU 302, the touch screen 304, the USB hub 306, the USB to serial converter 308 and a vital sign module 310. The device also provides connections for Wi-Fi 312, 3G network 314, GPRS 316 and GPS 318. A provision for Audio 320, MIC 322 and HDMI 324 is also provided. The control board 326 provides connections for keypad 328, LED/alarm 330 and power 332. Through USB to serial converter 308 the blood glucose 334, triglycerides 336 and total cholesterol 338 measuring devices are connected. Similarly through the USB hub 306, external device such as LAN 340, stethoscope 342 and 3G dangle 344 is connected.

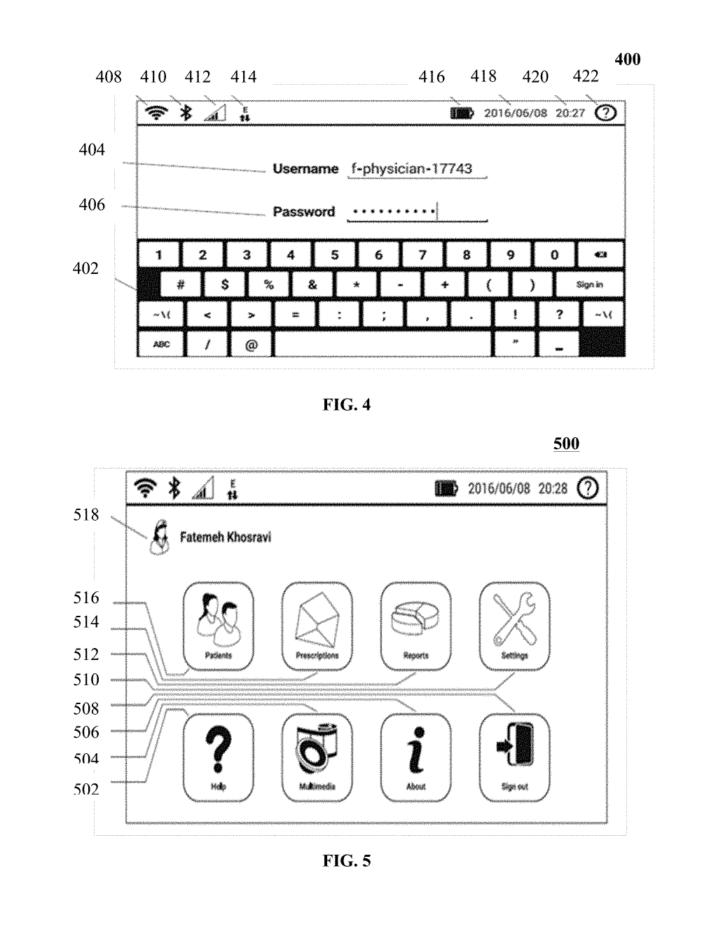

FIG. 4 illustrates a schematic representation of a login screen provided by the tele-monitoring health care device, according to one embodiment herein. With respect to FIG. 4, the login screen comprises a touchable keyboard 402 for entering username 404 and password 406 to login and enter the device's home screen. Username 404 and Password 406 for each device are defined by the service provider. The login screen further depicts enable or disable Wi-Fi icon (408), enable or disable Bluetooth icon (410), SIM card installed in the device status signal (412), enable or disable the 4G internet of SIM card icon (414), battery charging status (416), date (418), time (420) and a help icon (422).

FIG. 5 illustrates a schematic representation of a main/home screen provided by the tele-monitoring health care device, according to one embodiment herein. With respect to FIG. 5, the home screen comprises various options to be selected by the user such as device guide or help (502), display video or photo (504), general information about the device (506). After selecting the information option the user gets information about the hardware and software of the device, serial number, version, model and information on the NIBP module. The home screen further comprises a sign out option (508) for exiting the page and returning to the input screen and a device settings option (510). The user is allowed to change settings for Wi-Fi, mobile data, GPS and public settings by choosing this option. Public settings include device language, frequency notch filter, and brightness, contrast, server address, PCG sampling rate and the like. Reports and prescriptions are viewed by selecting options (512) and (514) respectively. By choosing option (516) the attendant enters the patient list page. The attendants name is shown on the display screen (518).

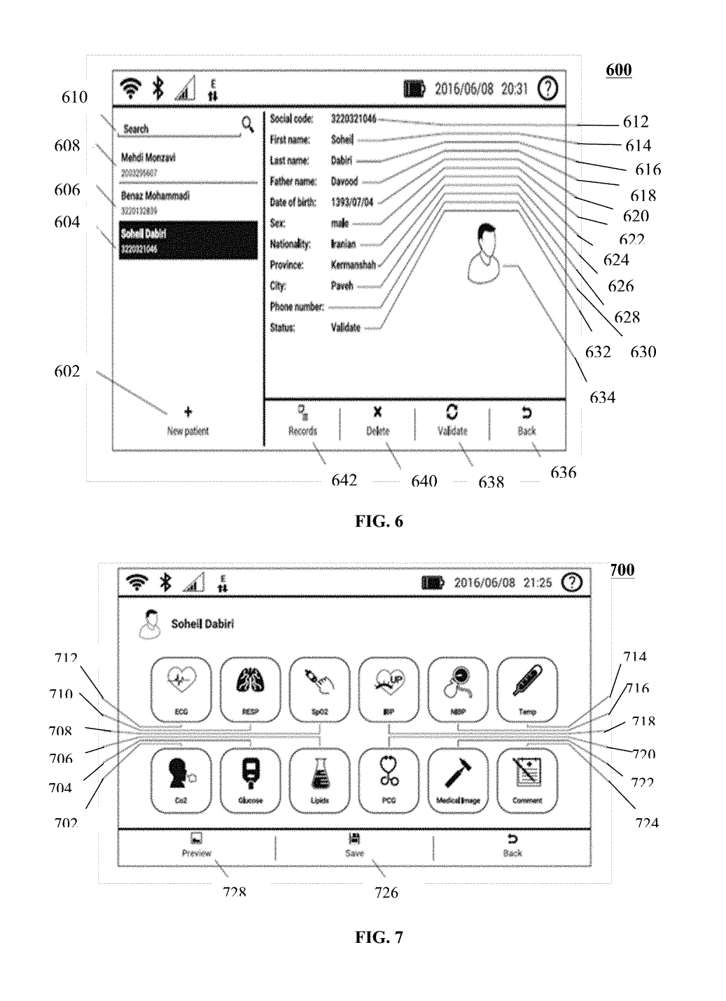

FIG. 6 illustrates a schematic representation of a patient information display screen provided by the tele-monitoring health care device, according to one embodiment herein. A new patient is added to the patient list 602. Tabs 604, 606 and 608 show the patients already listed with the device according to patient's visit order. Tab 610 is used for searching a particular patient name listed with the device. The patient's ID number is entered using tab 612, patient's name using tab 614, patient's family using tab 616, patient's father name using tab 618, patient's birth date using tab 620, patient's gender using tab 622, patient's nationality using tab 624, patient's birth place belonging to which state using tab 626, patient's birth city using tab 628 and patient's phone number using tab 630. Tab 632 is used for displaying whether the patient has visited before or not. The patient's photo that is saved before is displayed in tab 634. The back tab is 636. Tab 638 is used for validating the patient form existing list of patients when the device is connected to the network. To remove the patient from the list tab 640 is selected. Tab 642 illustrates patient records that have already been taken from patient.

FIG. 7 illustrates a schematic representation of a patient's vital signs measurement display screen provided by the tele-monitoring health care device, according to one embodiment herein. On choosing the tab 702 the user enters the main page of measuring CO₂. By choosing the tab 704 the user enters the main page of measuring the Glucose. By choosing the tab 706 the user enters the page of measuring the four types of fat in blood. By choosing the tab 708 the user enters the main page of measuring the blood oxygen saturation SPO₂. By choosing the tab 710 the user enters the page of measuring the Respiration of the patient. By choosing the tab 712 the user enters the main page of ECG. By choosing the tab 714 the user enters the patient's temperature page. By choosing the tabs 716 and 718 the user enters the NIBP and IBP page. By choosing the tabs 720 the user enters the PCG Recording Page. Tab 722 is used to display saved medical images. Tab 724 is used to write prescriptions/additional comments or interpreting tests by physicians for patients. Tab 726 is used to save the data taken from patients. Tab 728 is used to show the data taken from patients but which is not saved.

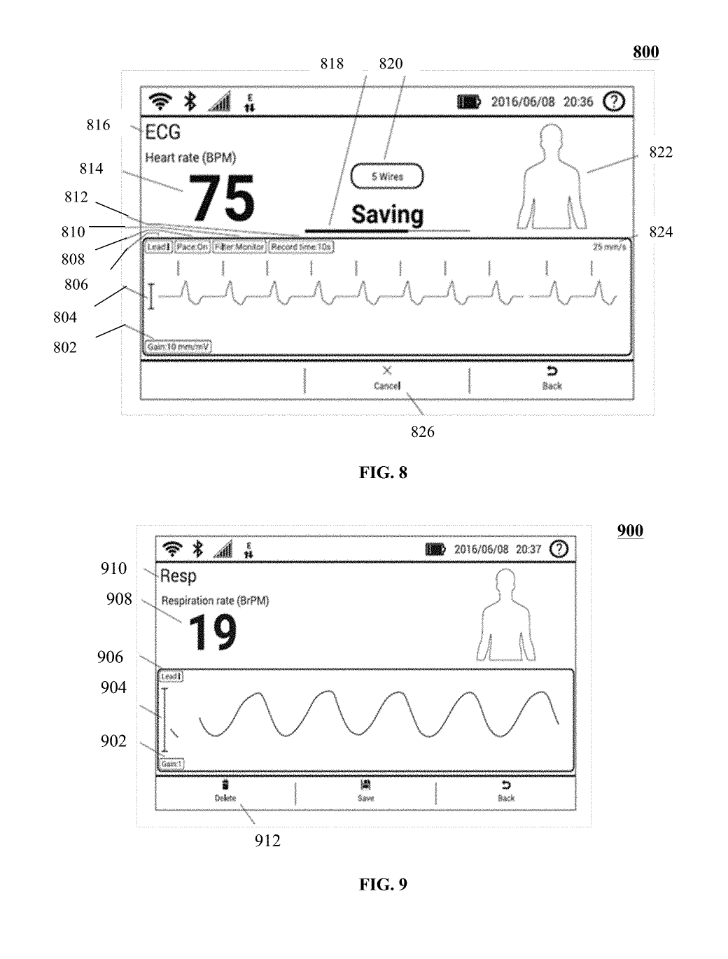

FIG. 8 illustrates a schematic representation of an ECG signal measurement and record display screen provided by the tele-monitoring health care device, according to one embodiment herein. The leads are connected to the patient for ECG and heart rate. Tab 802 shows the signal booster's rate. The signal booster rate is changed as needed. The signal booster's rate by default is 10 mm/mv and is configurable to 2.5, 5 or 20 mm/mv. Tab 804 shows Electrocardiogram (ECG) signal. Tab 806 displays the number of lead's used. Tab 808 shows presence of a pacemaker. When the patient has pacemaker the pacemaker tab is switches on to detect the pacemaker pulse. Tab 810 displays the kind of ECG filter which can be changed as needed. The default filter used is 0.05-100 HZ extended and can be changed to 0.5-40 Hz monitor or 0.5-25 Hz surgery. Tab 812 shows the recording time which is 10 S by default and can be changed to 20, 30, 40, 50 and 60 S. Tab 814 displays the heart rate of the patient. Tab 816 and 818 represent a measurable parameter and the page message. Tab 820 shows the number of wires used for connecting to the patient and by default 5 wires are used. The number of wires can be changed to 3 wires. Tab 822 shows the manner in which the leads are connected on the patient's body. Tab 824 represents the speed of ECG signal which is 25 mm/s by default. Tab 826 shows the cancel option.

FIG. 9 illustrates a schematic representation of a respiration measurement display screen provided by the tele-monitoring health care device, according to one embodiment herein. Tab 902 shows the respiration signal booster which can be changed by choosing this tab. By default the respiration signal booster is 1 and can be changed to 0.25, 0.5 and 2. Tab 904 displays a signal amplitude range. Tab 906 shows the number of leads and by choosing this tab the number of leads can be changed from 1 to 2. Tab 908 displays the respiration rate. Tab 910 represents a measurable parameter. Tab 912 shows delete tab and by choosing this tab the signal taken from the patient can be cleared.

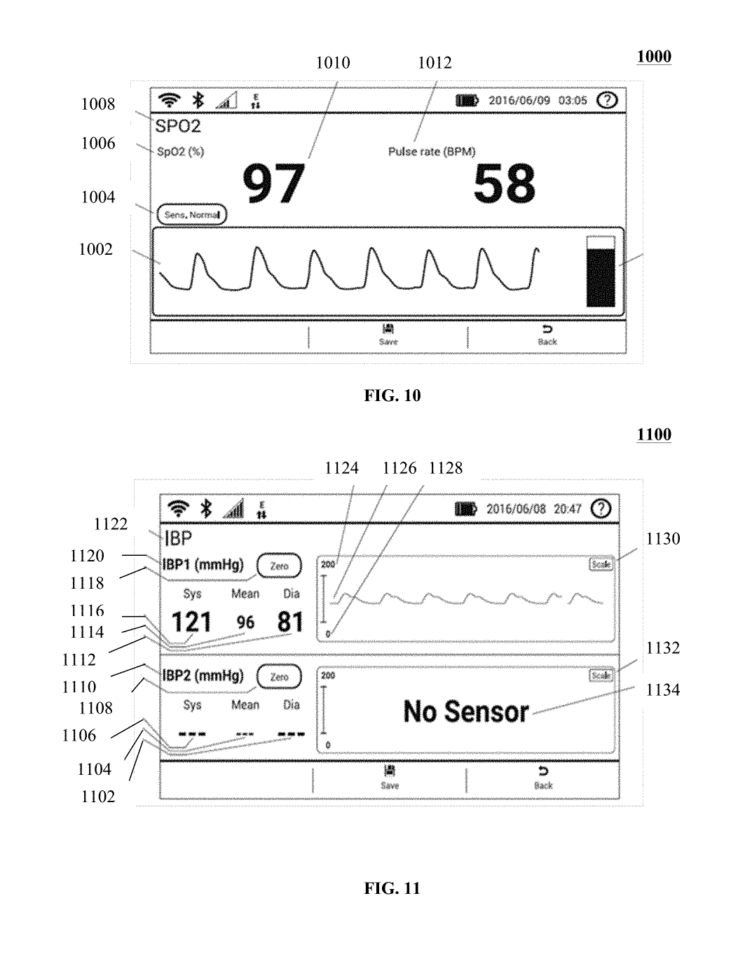

FIG. 10 illustrates a schematic representation of a signal blood oxygen saturation measurement display screen provided by the tele-monitoring health care device, according to one embodiment herein. To measure SPO₂ the sensors are connected to the patient. Tab 1002 shows the measured signal. Tab 1004 shows the sensitivity of SPO₂ and by default the sensitivity is set to normal which can be changed to high or low. Tab 1006 shows the SPO₂ as a percentage. Tab 1008 represents a parameter that is being measured. Tab 1010 and 1012 show the amount of SPO₂ and pulse rate measured by the SPO₂ module.

FIG. 11 illustrates a schematic representation of a invasive blood pressure measurement display screen provided by the tele-monitoring health care device, according to one embodiment herein. The device has two channels of invasive blood pressure measurement. To measure the invasive blood pressure a transducer and catheter is connected to the patient. Tabs 1102, 1104 and 1106 represent diastolic pressure, mean arterial pressure and systolic pressure which are measured by input number IBP2. By choosing tab 1108 the diastolic, mean arterial and systolic pressure of IBP2 are set to zero. Tab 1110 displays IBP input. Tabs 1112, 1114 and 1116 represent diastolic pressure, mean arterial pressure, systolic pressure measured using input number IBP1. By choosing tab 1118, the diastolic, mean arterial and systolic pressure of IBP2 are set to zero. Tab 1120 displays IBP number's input. Tab 1122 shows the measured parameters. Tab 1124 shows the maximum amplitude of the signal displayed for the IBP. Tab 1126 shows the measured IBP signal. Tab 1128 shows the minimum amplitude of the signal displayed for the IBP. Tab 1130 displays amplitude scale of the IBP1 waveform. By default, the scale is selected as ART (0-200) and can be changed to PAP (−10 to 380) or CVP (−10 to 30) or LAP (−10 to 40) or RAP (−10 to 30) or custom. Tab 1132 displays amplitude scale of the IBP2 waveform. Tab 1134 displays the message that no IBP2 sensors are attached.

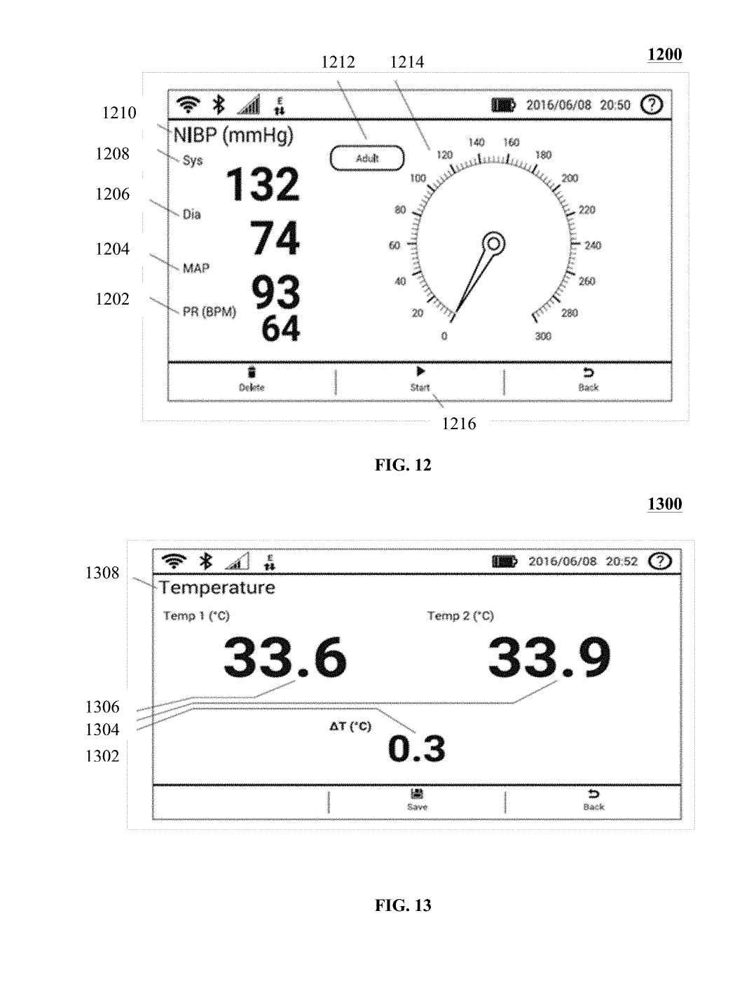

FIG. 12 illustrates a schematic representation of a noninvasive blood pressure measurement display screen provided by the tele-monitoring health care device, according to one embodiment herein. The cuffs are connected to the patient for measuring the parameters. Tab 1202 shows the heart rate of the patient. Tab 1204, 1206 and 1208 represent the mean arterial pressure, diastolic pressure and systolic pressure. Tab 1210 displays the measured parameter. Tab 1212 shows the type of patient such as a adult or a neonate. Tab 1214 displays situation of the wind filling the cuff attached to the patient. Tab 1216 shows the start tab and by choosing this tab the process of blood pressure measurement is started and the winds set the cuff to the initial size.

FIG. 13 illustrates a schematic representation of a patient's body temperature measurement display screen provided by the tele-monitoring health care device, according to one embodiment herein. The device has two channels for measuring temperature. Tab 1302 displays the temperature difference between the measured temperature between input number1 and number 2. Tabs 1304 and 1306 show the measured temperature using input number 2 (Temp2) and input number 1 (Temp1). Tab 1308 displays the measured parameter.

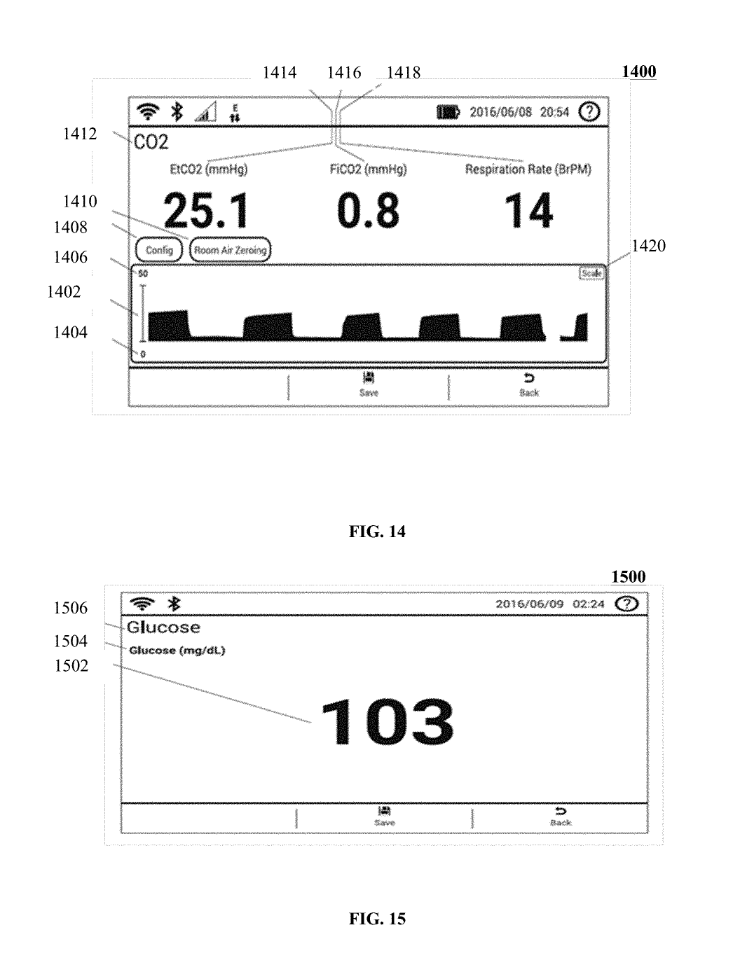

FIG. 14 illustrates a schematic representation of a CO₂ exhalation measurement display screen provided by the tele-monitoring health care device, according to one embodiment herein. To measure the parameter an oral adapter is used for the patient. Tab 1402 shows the waveform of the amount of CO₂ exhaled. Tabs 1404 and 1406 show the minimum and maximum amplitudes of the waveform. Tab 1408 provides a setting for capnograph and by choosing this tab the user can manually change density of oxygen and nitrous oxide. Density of oxygen by default is 0%-30% and can be changed to 30%-70% or 70%-100% and density of nitrous oxide can be selected between 0%-30% or 30%-70%. Also by choosing this tab the period of time Apnea error can be specified. Apnea error occurs when air is not found in the mouth adapter. By replacing the colored circle from left to right the time apnea error is changed from 20 to 60. Tab 1410 is used for setting zero reference used to measure CO₂ correctly. Tab 1412 displays the measured parameter. Tab 1414 and 1416 are used for displaying the parameters of EtCO₂ and FiCO₂. Tab 1418 shows the values of breathing rate measured using a capnograph module. Tab 1420 shows the amplitude of capnography waveform and by default it is 100 mmHg and can be changed to 50, 75, 150, 200 and 250.

FIG. 15 illustrates a schematic representation of a blood glucose measurement display screen provided by the tele-monitoring health care device, according to one embodiment herein. In the beginning, a blood glucose strip is attached to the glucose meter to measure this parameter. The stored information is transmitted through a cable to the main monitor and the information on this page is displayed. Tab 1502 shows the measured blood glucose levels. Tab 1504 displays unit of measurement of blood glucose. Tab 1506 shows the measured parameter.

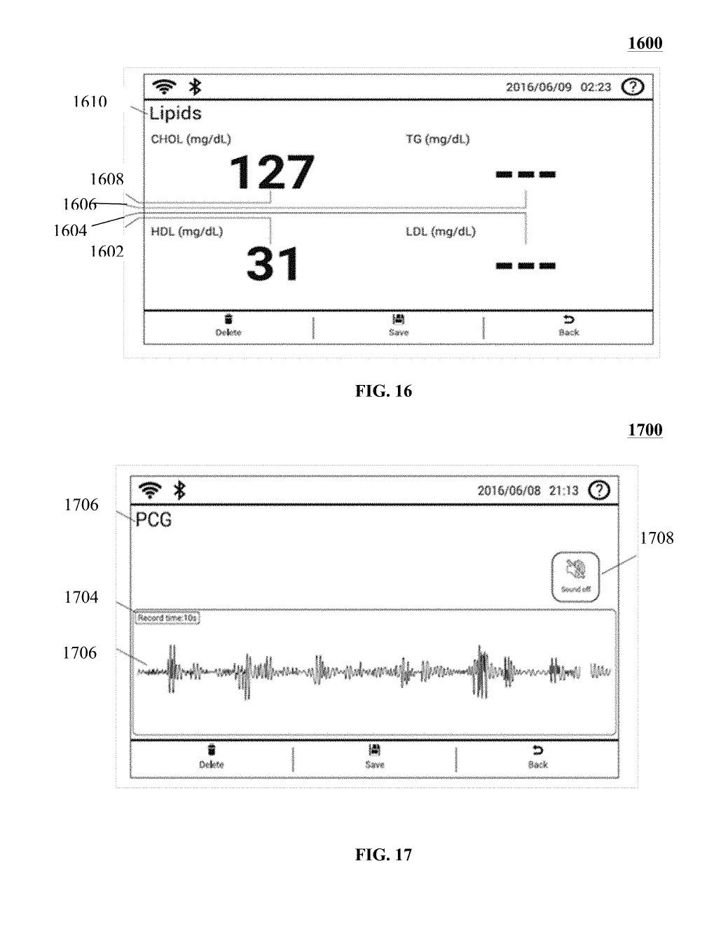

FIG. 16 illustrates a schematic representation of a four types of fats display screen provided by the tele-monitoring health care device, according to one embodiment herein. To measure this parameter a strip is inserted into the device in the beginning and then the measured information is transmitted through a cable to the main monitor. Tabs 1602 and 1604 show the HDL and LDL. Tabs 1606 and 1608 display Triglyceride and Cholesterol. Tab 1610 shows the measured parameter.

FIG. 17 illustrates a schematic representation of a PCG recording display screen provided by the tele-monitoring health care device, according to one embodiment herein. First stethoscope and headphones connected to the corresponding inputs on the monitor device and then the parameters are measured. Tab 1702 shows the Phonocardiogram signals. Tab 1704 determines the storage time of heartbeat which is 10 S by default and by choosing the tab this can be changed to 20, 30, 40, 50 and 60 S. Tab 1706 shows the measured parameter. By choosing the 1708 the sound is turned ON or OFF. Using this tab the heart beat sound is turned OFF and only the signal is viewed on the monitor and vice versa.

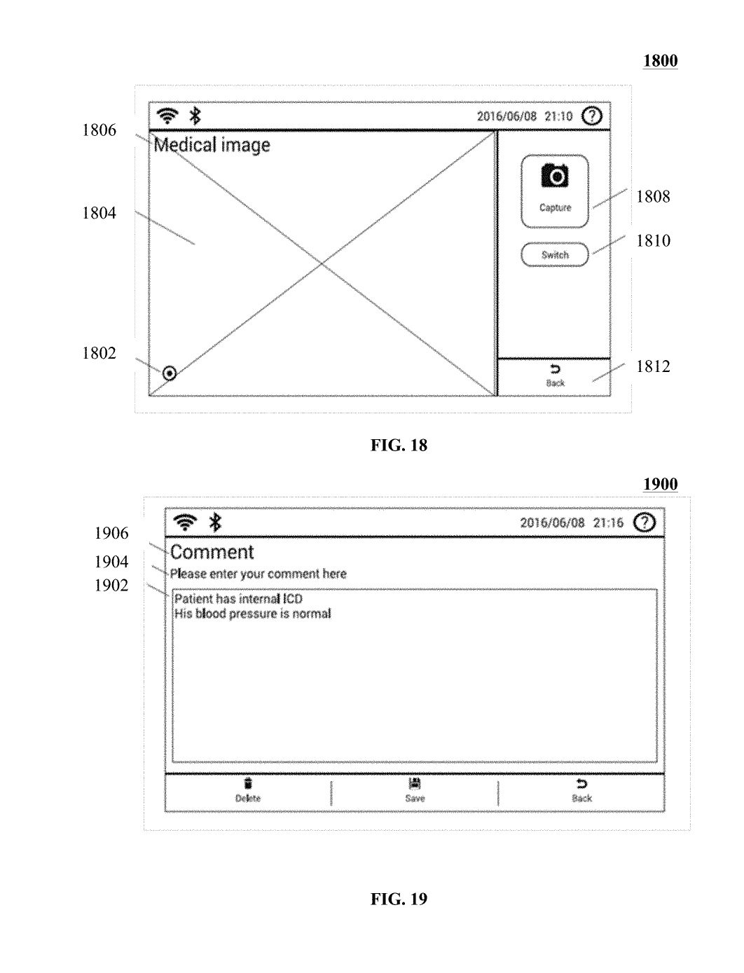

FIG. 18 illustrates a schematic representation of a medical image taken by the tele-monitoring health care device, according to one embodiment herein. The medical images are taken using a otoscope. The otoscope is connected to the device using a USB cable. Tab 1802 indicates the time at which the image is captured using otoscope. For enabling the user to take correct images, the colored circle moves from left part of screen to right indicating when required amount of time is spent and a proper image is available. Tab 1804 shows the image being displayed and saved. Tab 1806 shows the measured parameter. By choosing the tab 1808 the images can be saved and deleted. By choosing the tab 1810 the user can select between a webcam or otoscope. Tab 1812 is a back tab.

FIG. 19 illustrates a schematic representation of a comment/prescription provided by the tele-monitoring health care device, according to one embodiment herein. Using this page the doctor interprets the measured parameters of the patient and writes a prescription.

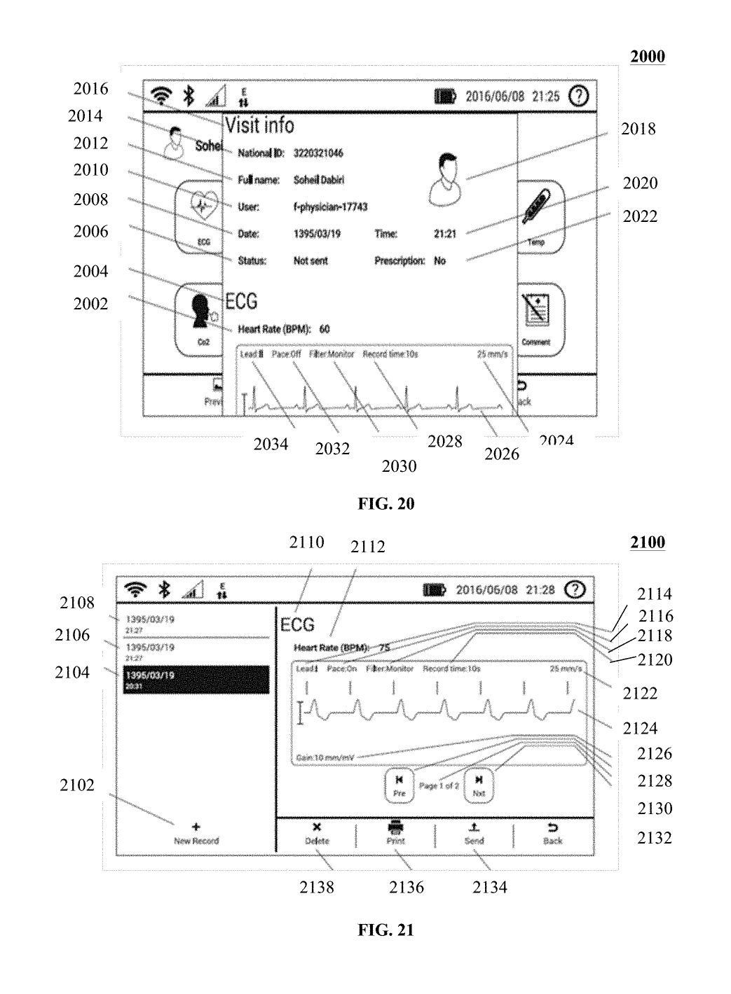

FIG. 20 illustrates a schematic representation of a page depicting all unsaved vital signs of the patient, according to one embodiment herein. Tab 2002 shows the heartbeat, tab 2004 displays the measured parameters, tab 2006 displays an option for transmission of data about whether the data should be transmitted to the central location or not. Tab 2008 shows the date of visit, tab 2010 displays the name of the doctor or user name. Tabs 2012 and 2014 show the patient's name and patient's ID number. Tab 2016 shows the title of the page and tab 2018 displays the patient's photo. Tab 2020 displays the time of visit. Tab 2022 determines whether the patient is prescribed or not. Tab 2024 shows ECG signal's rate, tab 2026 displays ECG signal taken from patient. Tab 2028 shows the saved signal's time and tab 2030 displays the kind of ECG signal. Tab 2032 shows the pacemaker condition. Tab 2034 displays the number of leads.

FIG. 21 illustrates a schematic representation of a page depicting data captured and stored from a patient as per date and time of visit, according to one embodiment herein. To enter the page the user has to select patient's name and then choose the record tab. Then all the data collected from the patient is displayed. By choosing the tab 642 form FIG. 6 (records) the user enters to FIG. 21 that shows the patient's list. Tab 2102 is used for adding new record. Tab 2104, 2106 and 2108 show the list of the patient's visit according date and time. Tab 2110 displays the measured parameter and the saved data. Tabs 2112, 2114, 2116, 2118, 2120, 2122, 2124 and 2126 are the ECG parameters as described earlier. By choosing tabs 2128 and 2130 the user can see the previous page and next page. Tab 2132 displays the number of pages visited which in turn show the measured number of parameters. Tab 2134 is used for sending the patient information to the central location/medical center. Tab 2136 is used for printing the displayed data. Tab 2138 is used for deleting the displayed parameter.

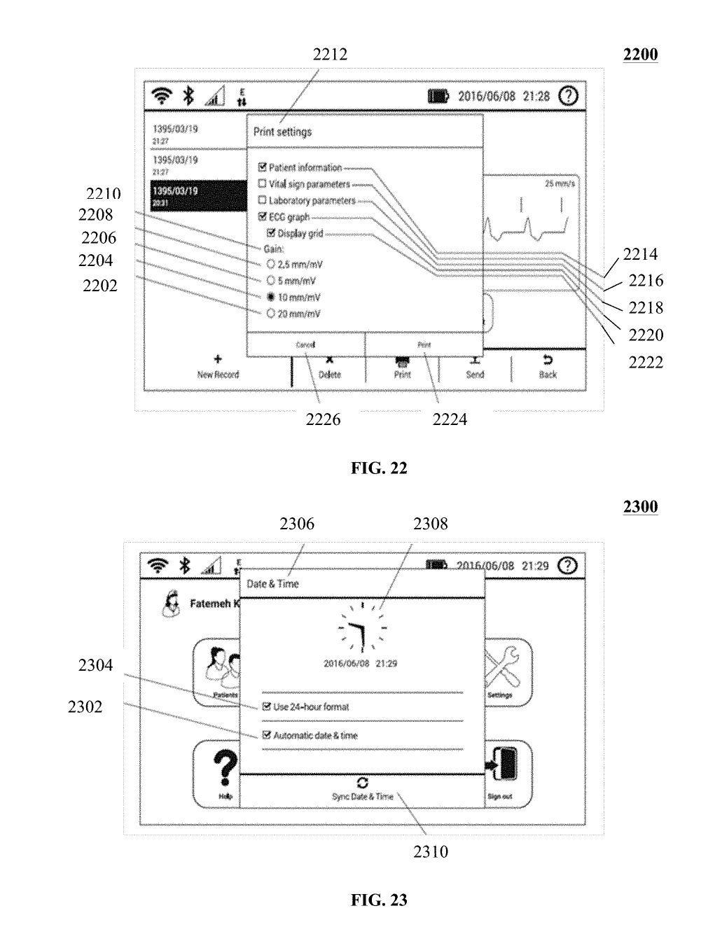

FIG. 22 illustrates a schematic representation of a screen depicting various print settings available in the tele-monitoring health care device, according to one embodiment herein. The user is allowed to determine data range and amplitude for printing. The user can select ECG signal rate between 20 mm/mv (2202), 10 mm/mv (2204), 5 mm/mv (2206) or 2.5 mm/mv (2208) to print. Tab 2210 shows ECG signal rate settings icon. Page title is shown using tab 2212. By choosing the tab 2214 user determines whether to print the patient's personal information or not. By choosing the tab 2216 user determines whether to print the patient's vital signs or not. By choosing tab 2218 user determines whether to print the patient's laboratory blood parameters or not. By choosing tab 2220 user determines whether to print the ECG graph or not. By choosing tab 2222 user determines whether to print the page's grid or not. Tabs 2224 and 2226 are used for proceeding with printing and for cancelling the printing.

FIG. 23 illustrates a schematic representation of a screen depicting date and time settings, according to one embodiment herein. By choosing date and time at the right top of the main/homepage the settings page appears. By choosing tab 2302 the device automatically adjusts the date and time using the network. Tab 2304 shows the clock in 24-hours format or can change it to A.M, P.M format. Tab 2306 shows the title of the page. Tab 2308 displays the current date and time that is set for the device. Tab 2310 shows the update date and time option by network when the device is connected to the internet.

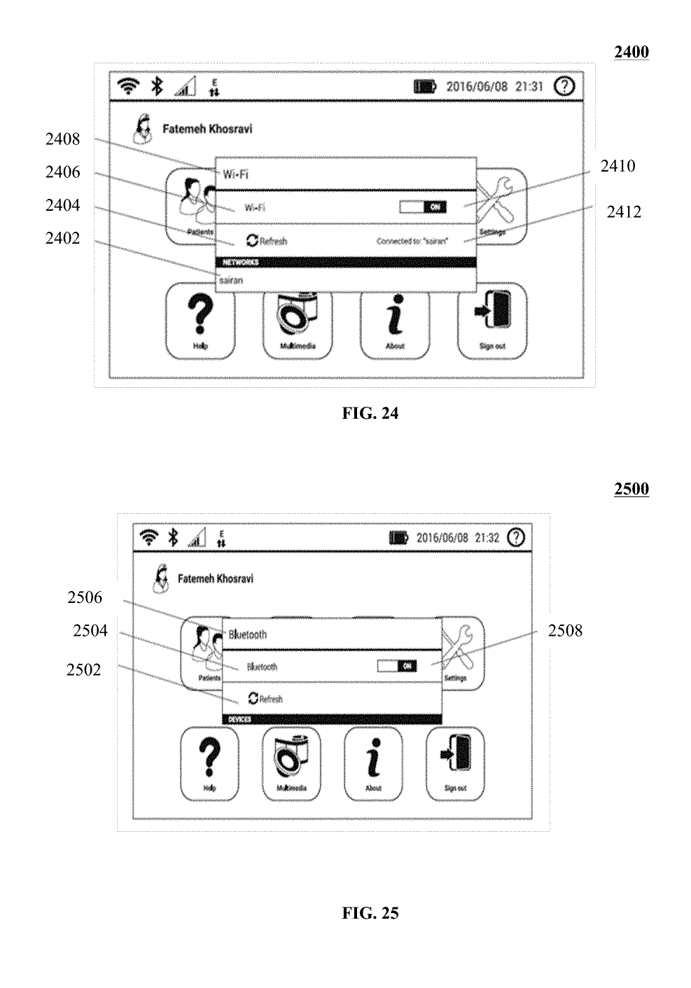

FIG. 24 illustrates a schematic representation of a screen depicting Wi-Fi settings, according to one embodiment herein. By choosing Wi-Fi option at the top left of the main/home page the Wi-Fi settings page is displayed. Tab 2402 shows the name of all the networks that are detected in the range of the device. Tab 2404 shows the search sign for network. Tab 2406 shows Wi-Fi key to turn on/off the Wi-FI. Tab 2408 shows the title of the page. Tab 2410 displays whether the Wi-Fi is on/off. Tab 2412 shows the name of the network that the device connects to.

FIG. 25 illustrates a schematic representation of a screen depicting power on/off and Bluetooth settings, according to one embodiment herein. By choosing Bluetooth tab at the left top of the main/home page the Bluetooth settings page appears. Tab 2502 shows the search sign for new device. Tab 2504 displays the Bluetooth key to on/off the Bluetooth. Tab 2506 displays the title of the page. Tab 2508 shows whether the Bluetooth is on or off.

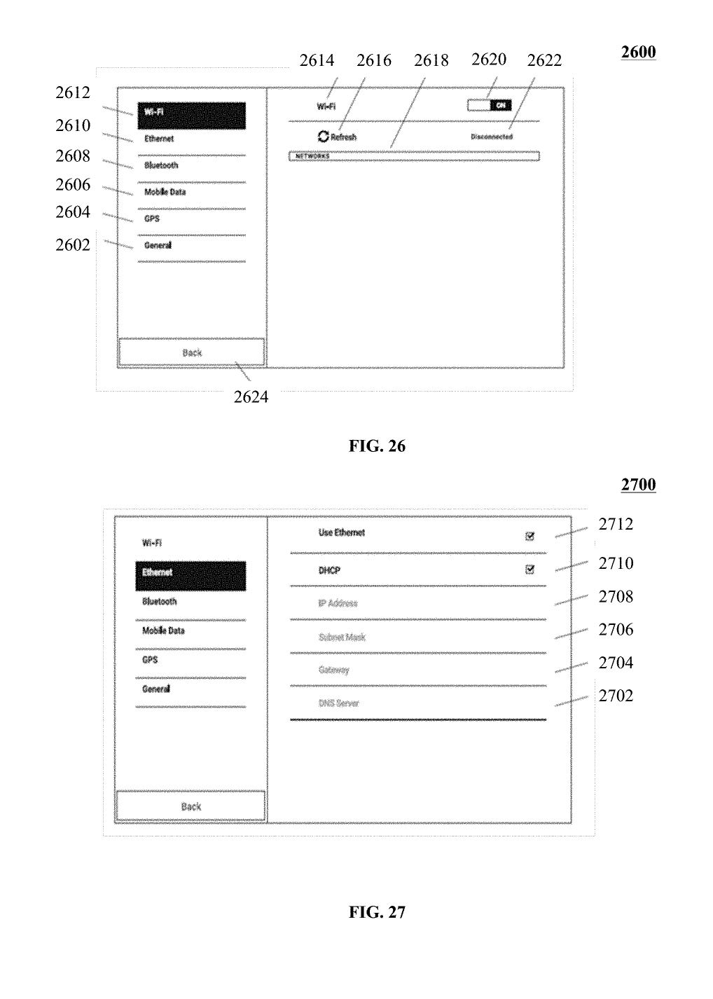

FIG. 26 illustrates a schematic representation of a screen depicting overall settings, according to one embodiment herein. By choosing the settings tab in mai/home page the user enters the settings page. Tabs 2602 and 2604 display the general settings and GPS settings of the device. Tabs 2606 and 2608 show the mobile data setting and Bluetooth setting. Tab 2610 display the Ethernet setting. Tab 2612 shows the Wi-Fi setting and by choosing this tab the Wi-Fi settings page appears. Tabs 2614 and 2616 display the title page and search sign to find a new network. Tab 2618 shows all the networks that have been detected in the range of the device. Tab 2620 displays the on/off status of the Wi-Fi. Tab 2622 shows whether the network is connected or disconnected. Tab 2624 is a back tab.

FIG. 27 illustrates a schematic representation of a screen depicting Ethernet settings, according to one embodiment herein. Tab 2702 displays whether the Ethernet is active or inactive. Tab 2704 displays whether the DHCP is active or inactive. By choosing tab 2706 user provides an IP address required for connectivity.

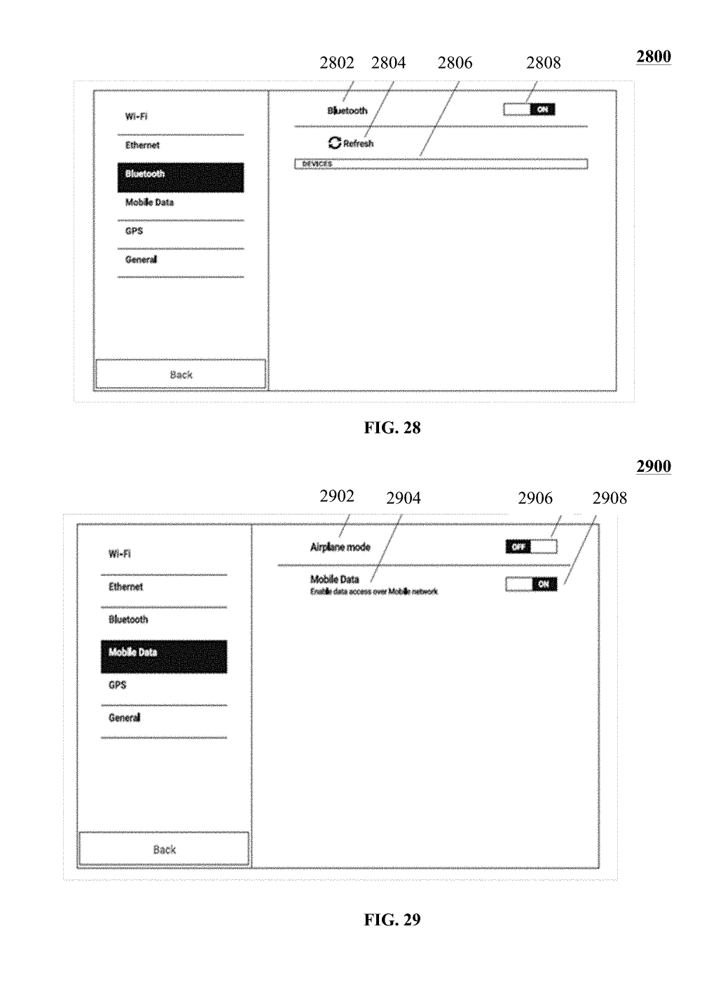

FIG. 28 illustrates a schematic representation of a screen depicting Bluetooth settings, according to one embodiment herein. Tabs 2802 and 2804 show the title of the page and search sign for new network. Tab 2806 displays the list of the devices that are detected in the range of the device. Tab 2808 shows that whether the device is On or Off.

FIG. 29 illustrates a schematic representation of a screen depicting mobile data settings, according to one embodiment herein. The SIM card is disabled by choosing tab 2902 by turning the airplane mode ON. Tab 2904 shows whether the mobile data is on or off. Tab 2906 is used for turning on/off the airplane mode. Tab 2908 displays the on/off mobile data icon.

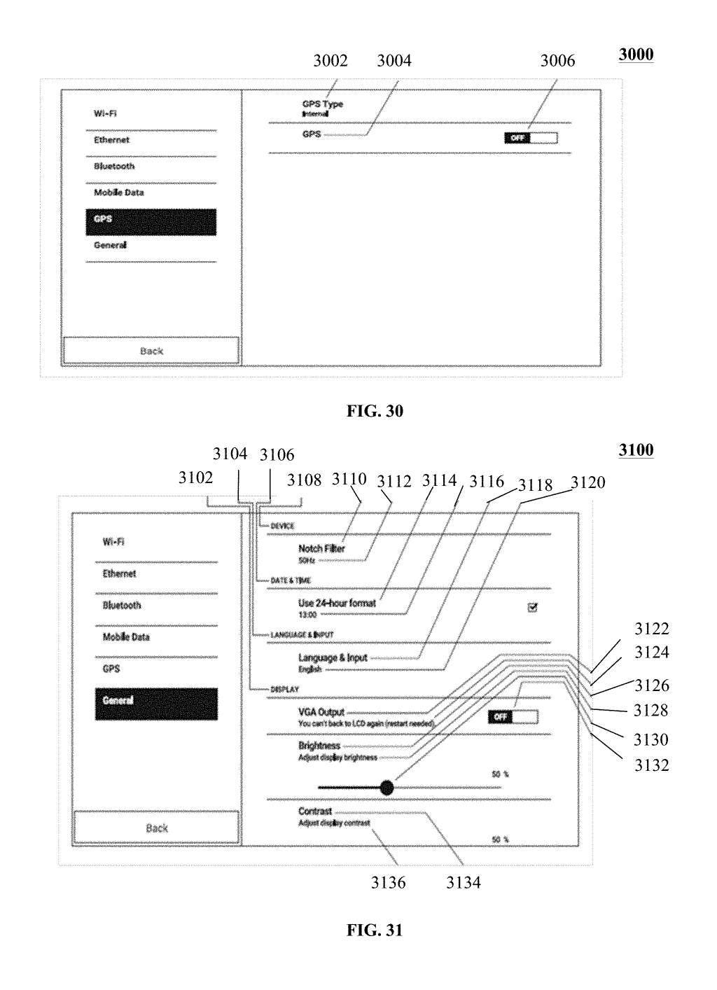

FIG. 30 illustrates a schematic representation of a screen depicting GPS settings, according to one embodiment herein. Tab 3002 shows the type of GPS. The device uses either internal or external GPS. Tab 3004 shows the GPS sign. Tab 3006 shows the on/off GPS icon.

FIG. 31 illustrates a schematic representation of a screen depicting general settings, according to one embodiment herein. Tab 3102 represents the monitor settings. Tab 3104 shows the device language or keyboard language. Tab 3106 displays the date and time settings. Tab 3108 shows the monitor equipment settings. Tab 3110 represents the notch filter frequency settings. A notch filter is a band-stop filter with a narrow stopband. A band-stop filter is a filter that passes most frequencies unaltered, but attenuates those in a specific range to very low levels. Tab 3112 shows the adjusted notch filter frequency which is by default 50 HZ and can be changed to 60 HZ or turned off. Tab 3114 shows the clock as 24-hours format or can be changed to A.M, P.M format. Tab 3116 shows the time. Tab 3118 represents the device language settings and keyboard language which is either English or Persian. Tab 3120 shows the device adjusted language which is by default English. Tab 3122 shows VGA output settings. The maximum resolution is 1536×2048. Tab 3124 shows the message that VGA output is using and picture doesn't show anymore unless the device is reset. To turn on/off VGA output tab 3126 is used. By choosing tab 3128 the monitor brightness is adjusted. Tab 3130 displays the message of the icon. Tab 3132 displays the percentage of monitor brightness and by replacing the colored circle from left to right the brightness is changed. Tab 3134 shows the monitor contrast settings page. Tab 3136 displays the message of the icon.

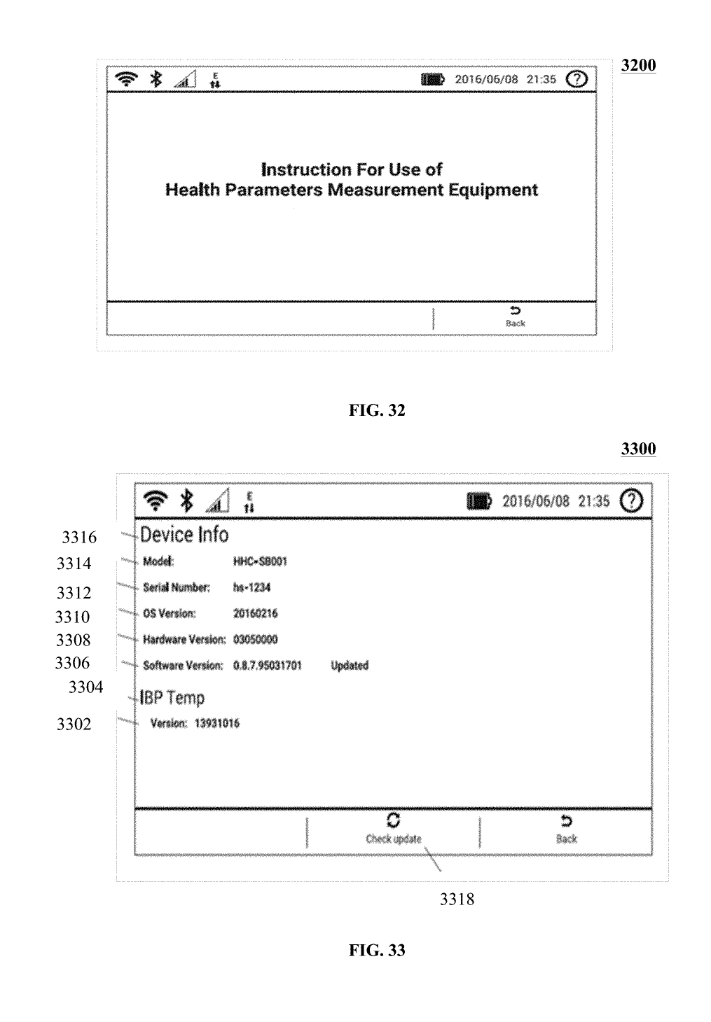

FIG. 32 illustrates a schematic representation of a help screen, according to one embodiment herein. By choosing the help icon in the main/home page the user goes to device help page.

FIG. 33 illustrates a schematic representation of a device information screen, according to one embodiment herein. By choosing the about tab device enters the device information page. All the device information on this page is listed along with information about the modules. Tab 3302 shows the version of IBP module software. Tab 3304 displays the kind of module. Tab 3306 represents the version of main software of device. Tab 3308 shows the version of device hardware. Tab 3310 displays the version of operating system. Tab 3312 shows the specific serial number of device. Tab 3314 shows the device model. Tab 3316 displays the monitor's name. By choosing tab 3318 the device checks the software update by connecting to network.

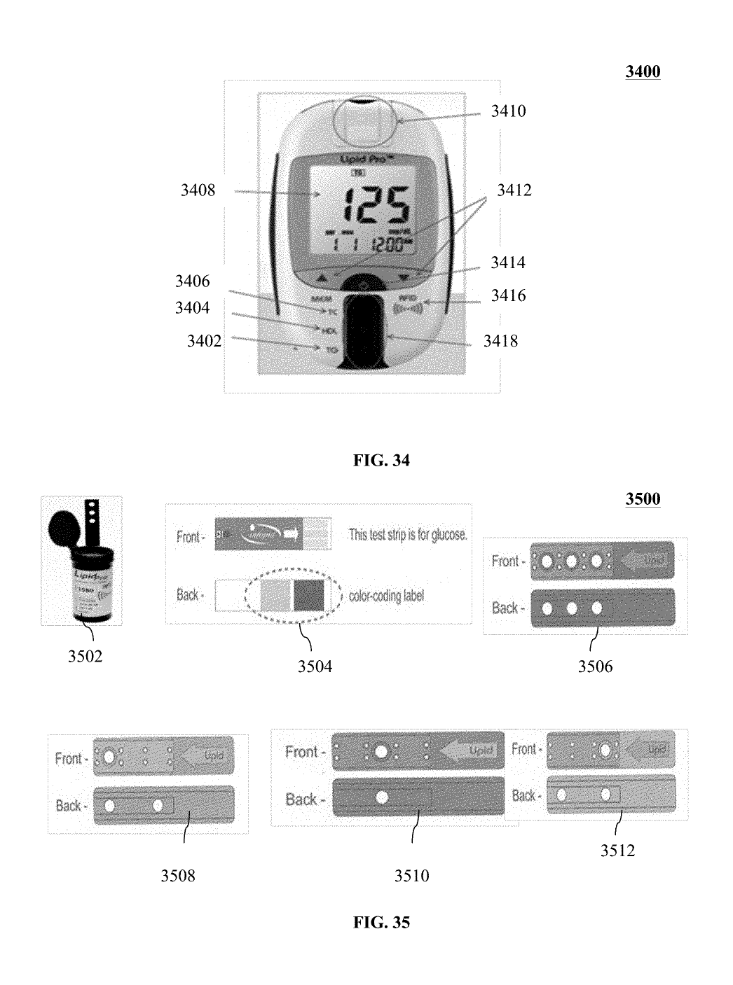

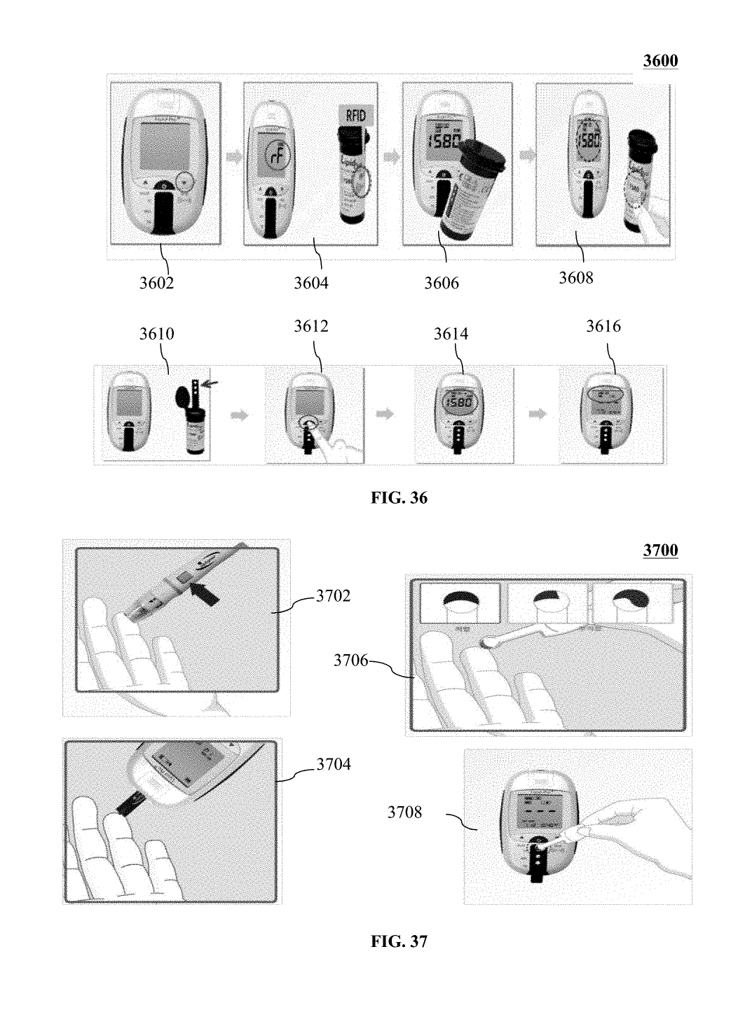

FIG. 34 illustrates a blood parameter measuring device provided with the portable health care tele-monitoring device, according to one embodiment herein. The blood parameter measuring device is used for measuring plurality of blood parameters of the patient such as cholesterol, HDL (high-density lipoprotein), LDL (low-density lipoprotein), triglycerides and blood glucose. The blood parameter measuring device is connected to the tele-monitoring device through the blood parameter measuring device port. The blood parameter measuring device is switched on and then the download key is pressed at the bottom of the device to display all the measured parameters on the display unit. The various parts of the blood parameter measuring device comprise triglyceride 3402, HDL-cholesterol 3404, total cholesterol 3406, LCD display 3408, glucose test part 3410, up and down button 3412, mode button 3414, RFID tag contact point 3416 and cholesterol test part 3418. The blood parameter measuring device needs various types of test strips for measuring fat and other measuring blood parameters as shown in FIG. 35. In one example embodiment test stripes with mark Lipidpro (3502) are used. The various strips include blood glucose stripes 3504, blood fat stripes 3506, total cholesterol test stripes 3508, HDL cholesterol test stripes 3510 and triglyceride test stripes 3512.

FIG. 36 illustrates the steps followed in code testing for the blood parameter measuring device, according to one embodiment herein. With respect to FIG. 36, a button in the top right corner of the device is pressed for at least 3 seconds (3602). A symbol “rF” appears on the monitor of the device (3604). The user holds a RFID chip for 5 seconds in the range of the device (3606). The RFID code on the box containing the test stripes is displayed on the monitor (3608). Further, the test stripes are inserted into their designated place as depicted in steps 3610, 3612, 3614 and 3616 on successful checking of RFID code on the device and the box containing the test stripes.

FIG. 37 illustrates the steps followed in measuring plurality of blood parameters using the blood parameter measurement device, according to one embodiment herein. With respect to FIG. 37, a special needle is inserted into the finger of the patient for making a small prick to collect blood (3702). The blood is poured on the shield on the stripe in a manner that the shield is full of blood (3704). The device displays the blood glucose test result in about 3 seconds. Similarly, for measuring blood lipid profile, blood is collected from the finger using a thin rod (3706). Then the rod is inserted into a designated area in the blood parameter measuring device (3708) and the results are observed after 3 seconds.

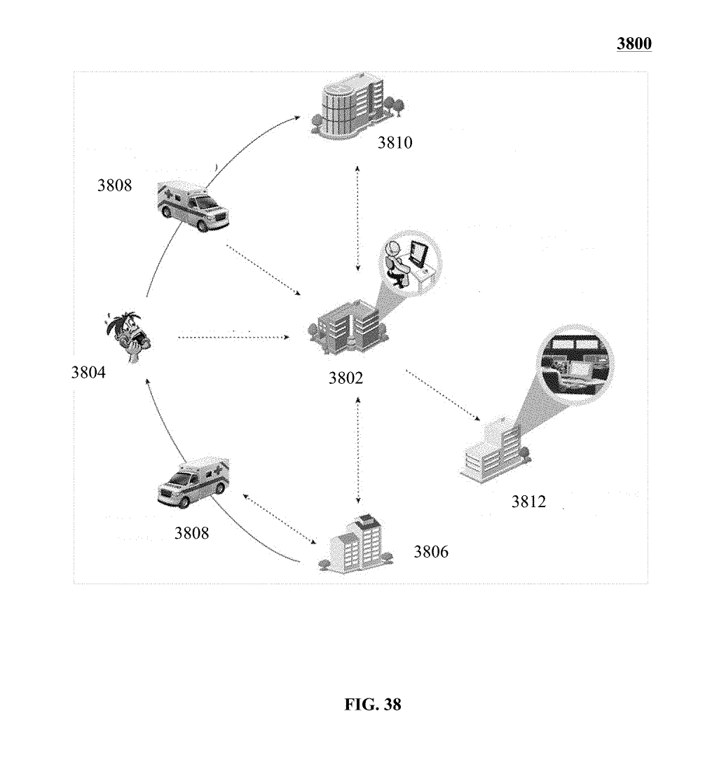

FIG. 38 illustrates a manner in which the portable health care tele-monitoring devices are used in ambulances, according to one embodiment herein. With respect to FIG. 38, an emergency call is received at the control centre (3802) for a patient (3804) in need. The control centre (3802) then contacts the base station (3806) about the situation. The base station (3806) forwards the patient location to a nearby ambulance (3808). Once the patient boards the ambulance, then the patient vital signs as well as blood parameters are checked using the portable health care tele-monitoring device placed inside the ambulance. The patient information along with the measured vital signs is transmitted to the control centre (3802) using the tele-monitoring device. The patient information and the vital signs data is further transmitted to a hospital (3810) so that the medical staff from the hospital does all the preparations to provide necessary medical care for the patient. The ambulance location details are also sent to an emergency operation centre (EOC) (3812) for monitoring ambulance location and for specifying most optimal route with minimal traffic to enable smooth ambulance transit.

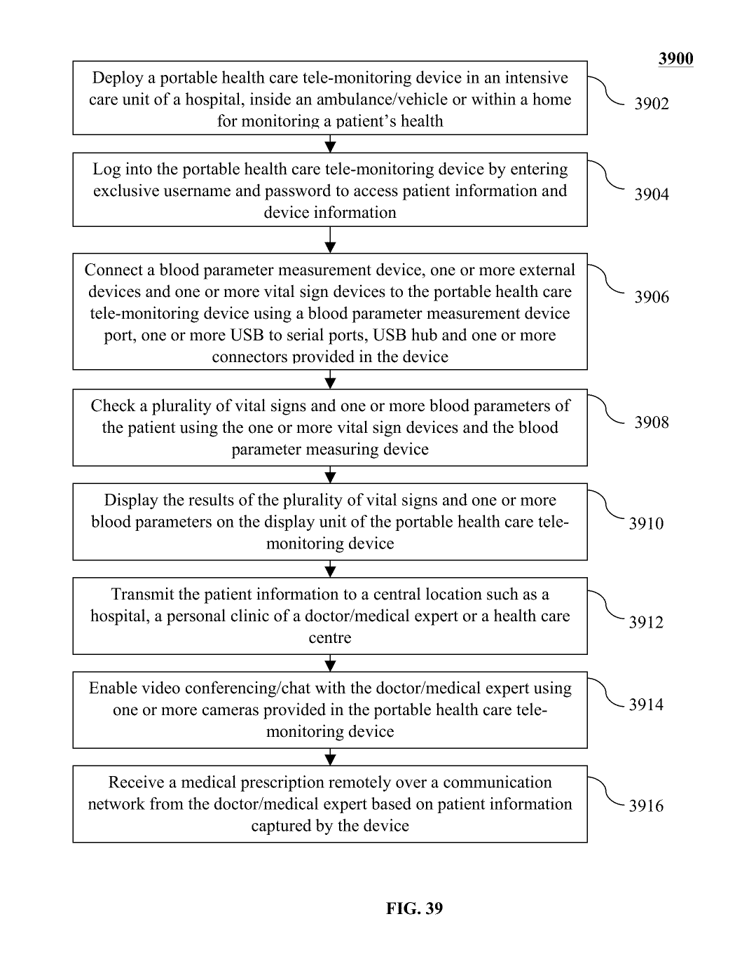

FIG. 39 illustrates a flow chart depicting a method of using the portable health care tele-monitoring device, according to one embodiment herein. The method comprises the steps of deploying a portable health care tele-monitoring device in an intensive care unit of a hospital, inside an ambulance/vehicle or within a home for monitoring a patient's health (3902). The method further comprises logging in the portable health care tele-monitoring device by entering exclusive username and password to access patient information and device information (3904). The patient information comprises one or more personal details of the patient, one or more health records of the patient and one or more real time medical parameters of the patient. The device information comprises a plurality of device configurable parameters and a instruction manual for operating the device. The method still further comprises connecting a blood parameter measurement device, one or more external devices and one or more vital sign devices to the portable health care tele-monitoring device using a blood parameter measurement device port, one or more USB to serial ports, USB hub and one or more connectors provided in the device (3906). A plurality of vital signs and one or more blood parameters of the patient are checked using the one or more vital sign devices and the blood parameter measuring device (3908). The results of the plurality of vital signs and one or more blood parameters are displayed on the display unit of the portable health care tele-monitoring device (3910). The patient information is transmitted to a central location (3912). The central location comprises a hospital, a personal clinic of a doctor/medical expert or a health care centre. Video conferencing/chat is enabled with the doctor/medical expert using one or more cameras provided in the portable health care tele-monitoring device (3914). A medical prescription is received remotely over a communication network from the doctor/medical expert based on patient information captured by the device (3916)

Therefore, the portable tele-monitoring health care device facilitates real time monitoring of patients health. The device also helps in measuring vital signs and blood parameters of the patient through various ports and connectors provided in the device for connecting vital sign devices and blood parameter measurement device. A physical copy of the patient medical records is printed using the thermal printer provided in the device. A provision for connecting external printer is also provided in the device. The patient information is transmitted to a doctor/medical expert using the device to receive a medical prescription/advice in real time.

Further, the patient is also facilitated with a user interface to chat with the doctor on a need basis. The device comprises a location detection module for tracking real time location when placed in an ambulance. This helps in determining the exact location and time of arrival of the ambulance. Thus, the user is enabled to interact with the central location through vocal or video conferencing and physicians/doctors access overall situation of the patient. The device settings are changed using the help manual provided in the device to help the user. The device is configured to measure the vital signs and physiologic parameter such as ECG, SpO₂, Respiration, NIBP, IBP, Temperature and CO₂ and blood factor such as PCG, Glucose, Cholesterol, Triglycerides, HDL. The device is connected with the central location using Bluetooth or Wi-Fi.

Thus, some of the important benefits offered by the portable tele-monitoring device health care comprise user friendly GUI, easy health check and self health management, video telephony and consultation, portable design and light weight, multiple blood parameter checks, reduced physician fees and cost of medicine, constant health consulting and education to patients, early diagnosis and treatment, reduced burden of morbidity, flexibility and choice of treatment, easy access to expertise of medical specialists, personalized health care possibility, less dependent on time and place, medical advice offering and patient empowerment. The device is used in various applications such as fitness centers, pharmacy, hospitals, homes, military, clinics, transportation and the like.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be the to fall there between.

What is claimed is:

1. A portable health care tele-monitoring device, the device comprising:
a housing comprises a base portion, a top portion, a first lateral side and a second lateral side;
a first panel provided in the base portion of the housing, and wherein the first panel is configured for accommodating a thermal printer, a printer output port, a blood parameter measurement device and a blood parameter measurement device port, and wherein the thermal printer is configured for printing a plurality of vital signs of the patient, and wherein the printer output port is configured for connecting external printer, and wherein the blood parameter measurement device is configured for measuring a plurality of blood parameters of the patient, and wherein the plurality of blood parameters of the patient comprise cholesterol, HDL (high-density lipoprotein), LDL (low-density lipoprotein), triglycerides and blood glucose;
a second panel provided in the first lateral side of the housing, and wherein the second panel is configured for accommodating a headphone connector, a Video Graphics Array (VGA) connector, one or more USB to serial ports and a USB hub, and wherein the one or more USB to serial ports and the USB hub are configured for connecting one or more external devices to the tele-monitoring device, and wherein the one of more external devices comprise a stethoscope, a telecommunication device and a networking device;
a third panel provided in the second lateral side of the housing, and wherein the third panel is configured for accommodating one or more connectors for connecting one or more vital sign devices to the tele-monitoring device, and wherein one or more vital sign devices are configure for measuring the plurality of vital signs of the patient, and wherein the plurality of vital signs of the patient comprise electro cardio gram (ECG), blood oxygen saturation level (SPO₂) level, respiration rate, non-invasive blood pressure (NIBP), invasive blood pressure (IBP), temperature, carbon dioxide (CO₂) and phonocardiogram (PCG);
one or more cameras provided in the top portion of the housing, and wherein the one or more cameras are configured for capturing one or more images of the patient or for facilitating real time video conferencing between the patient and a doctor; and
a central processing unit (CPU) provided within the housing, and wherein the CPU is configured for controlling operation of the portable health care tele-monitoring device and for storing the list of plurality of patients, patient information and device information in a database;
a display unit configured for displaying a list of plurality of patients registered with the device, patient information and device information, and wherein the patient information comprises one or more personal details of the patient, one or more health records of the patient and one or more real time medical parameters of the patient, and wherein the device information comprises a plurality of device configurable parameters and an instruction manual for operating the device, and wherein the display unit comprises a LCD/LED screen enclosed within the housing.

2. The device according to claim 1, wherein the device is further configured for enabling a user/attendant/medical service provider to access the device by logging into the device using an exclusive username and password, and wherein the one or more users/attendants/service providers are registered with the device using the username and password.

3. The device according to claim 1 further comprises one or more tabs provided in the first panel, and wherein the one or more tabs are configured for performing one or more operations of the device when the one or more tabs are pressed by an attendant, and wherein the one or more operations comprise powering ON/OFF the device, displaying a main menu page of the device, displaying a previously accessed page by the attendant, downloading and displaying one or more real time medical parameters of the patient and controlling the device volume.

4. The device according to claim 1 further comprises a connectivity module configured for communicatively connecting the device and a central location or a central server, and wherein the central server is provided in the central location, and wherein the central location is any one of a hospital, a personal clinic of a doctor/medical expert or a health care centre, and wherein the communication connection between the device and the central location or central server is established using Wi-Fi, Bluetooth or GPRS based connectivity.

5. The device according to claim 1 further comprises a control board configured for communicatively connecting the device with an external keypad, LED indicator/alarm, a power source and a global positioning system.

6. The device according to claim 1 further comprises a connector port configured for connecting the device with a high-definition multimedia interface (HDMI) and a microphone (mic).

7. The device according to claim 1, wherein the device is configured for use in an intensive care unit of a hospital, inside an ambulance/vehicle or within a home.

8. The device according to claim 1 further comprises a location detection module, and wherein the location detection module is configured for detecting a real time location of the device, and wherein the real time location of the device is transmitted to the central location or the central server.

9. The device according to claim 1, wherein the device is further configured for enabling the doctors/medical experts to write/share a prescription remotely based on the one or more health records of the patient and the one or more real time medical parameters of the patient monitored using the monitoring device.

10. The device according to claim 1 further comprises a touch screen keypad displayed in the display unit, and wherein the touch screen keypad is configured for enabling the user/attendant/medical service provider to log-in into the device using the exclusive username and password.

11. The device according to claim 1, further comprises a user interface to enable the patient/user for communicating with the doctor/medical expert whenever needed.

12. A method of using a portable health care tele-monitoring device, the method comprising the steps of:
deploying a portable health care tele-monitoring device in an intensive care unit of a hospital, inside an ambulance/vehicle or within a home for monitoring a patient's health;
logging into the portable health care tele-monitoring device by entering username and password to access patient information and device information, and wherein the patient information comprises one or more personal details of the patient, one or more health records of the patient and one or more real time medical parameters of the patient, and wherein the device information comprises a plurality of device configurable parameters and a instruction manual for operating the device;
communicatively connecting a blood parameter measurement device, one or more external devices and one or more vital sign devices to the portable health care tele-monitoring device using a blood parameter measurement device port, one or more USB to serial ports, USB hub and one or more connectors provided in the device;
checking a plurality of vital signs and one or more blood parameters of the patient using the one or more vital sign devices and the blood parameter measuring device;
displaying the results of the plurality of vital signs and one or more blood parameters on the display unit of the portable health care tele-monitoring device;
transmitting the patient information to a central location, and wherein the central location comprises a hospital, a personal clinic of a doctor/medical expert or a health care center;
enabling video conferencing/chat with the doctor/medical expert using one or more cameras provided in the portable health care tele-monitoring device; and
receiving a medical prescription remotely over a communication network from the doctor/medical expert based on patient information captured by the device.

13. The method according to claim 12, further comprises tracking a location of the ambulance provided with the portable health care tele-monitoring device in real time using a location determination module provided in the portable health care tele-monitoring device.

14. The method according to claim 12, further comprises providing a user interface to enable the patient/user for communicating with the doctor/medical expert whenever needed.

15. The method according to claim 12, further comprises optionally printing a physical copy of the plurality of vital signs and one or more blood parameters using a thermal printer or external printer attached with the portable health care tele-monitoring device.
